(19) United States
(12) Patent Application Publication
(10) Pub. No.: US 2013/0261464 A1
(43) Pub. Date: Oct. 3, 2013

(54) CONFORMAL FETAL HEART MONITOR AND SYSTEM FOR USE THEREWITH

(71) Applicants: Rahul Singh, Carlsbad, CA (US); Martin Culjat, Los Angeles, CA (US); Richard Brand Caso, Mission Viejo, CA (US); Jim Selevan, Laguna Beach, CA (US)

(72) Inventors: Rahul Singh, Carlsbad, CA (US); Martin Culjat, Los Angeles, CA (US); Richard Brand Caso, Mission Viejo, CA (US); Jim Selevan, Laguna Beach, CA (US)

(21) Appl. No.: 13/435,092

(22) Filed: Mar. 30, 2012

(51) Int. Cl. A61B 8/02

(52) U.S. Cl. 600/453

(57) ABSTRACT

A conformal fetal heart monitor includes a flexible substrate and a ultrasonic transducer array, coupled to the flexible substrate, that generates an ultrasonic transmission signal over a sequence of beam angles, that receives an ultrasonic return signal, and that generates an ultrasonic receive signal in response to the ultrasonic return signal. A transducer interface couples the ultrasonic receive signal to a processing system. A flexible housing that houses the flexible substrate, the ultrasonic transducer array and the transducer interface, wherein the flexible housing flexibly, when deployed, conforms to the surface of an abdomen of a patient.

### STATEMENT REGARDING FEDERALLY SPONSORED RESEARCH OR DEVELOPMENT

Portions of the present invention were funded by the National Institutes of Health (NIH), NIH Award #1R43HD069079-01.

### CROSS REFERENCE TO RELATED PATENTS

NOT APPLICABLE

### INCORPORATION-BY-REFERENCE OF MATERIAL SUBMITTED ON A COMPACT DISC

NOT APPLICABLE

### BACKGROUND OF THE INVENTION

1. Technical Field of the Invention

This invention relates generally to sensors for monitoring the heartbeat in a patient.

2. Description of Related Art

External fetal heart monitors (FHMs) allow for intermittent or continuous monitoring of fetal status both before and during labor, and reduce medical costs by allowing fewer nurses or midwives to manage a larger number of patients. However, current external FHMs are cumbersome in design, leading a significant negative impact on patient comfort and mobility. External FHMs are rigid, require straps to maintain their position on the abdomen, and are typically tethered to a large fetal monitoring system. During continuous monitoring, the patient's movements and positions are considerably restricted, so as not to perturb the position of the FHM and compromise the fidelity of the fetal heart rate signal.

Movement during the monitoring can result in a loss of the fetal heart rate signal and lead to false alarms and unnecessary patient anxiety. These false alarms can also result in increased medical costs, due to the need for nurses or midwives to frequently reposition the transducer on the abdomen.

Further disadvantages of conventional approaches will be evident to one skilled in the art when presented the disclosure that follows.

### BRIEF SUMMARY OF THE INVENTION

The present invention is directed to apparatus and methods of operation that are further described in the following Brief Description of the Drawings, the Detailed Description of the Invention, and the claims. The features and advantages of the present invention will become apparent from the following detailed description of the invention made with reference to the accompanying drawings.

### BRIEF DESCRIPTION OF THE SEVERAL VIEWS OF THE DRAWING(S)

FIG. 1 is a block diagram representation of an embodiment of fetal heart monitoring system including conformal fetal heart monitor 100 and personal computer 20 in accordance with the present invention;

FIG. 2 is a block diagram representation of an embodiment of fetal heart monitoring system including conformal fetal heart monitor 100 and processing system 200 in accordance with the present invention;

FIG. 3 a block diagram representation of a conformal fetal heart monitor 100 in accordance with the present invention;

FIG. 4 is a top view of an embodiment of conformal fetal heart monitor 101 in accordance with the present invention;

FIG. 5 is a top view of an embodiment of conformal fetal heart monitor 102 in accordance with the present invention;

FIG. 6 is a back view of an embodiment of a flexible housing 36 in accordance with the present invention;

FIG. 7 is a top view of another embodiment of a flexible housing 44 in accordance with the present invention;

FIG. 8 is a side view of another embodiment of a flexible housing 46 in accordance with the present invention; and

FIGS. 9-11 are top views of other embodiments of conformal fetal heart monitor 101 in accordance with the present invention.

### DETAILED DESCRIPTION OF THE INVENTION

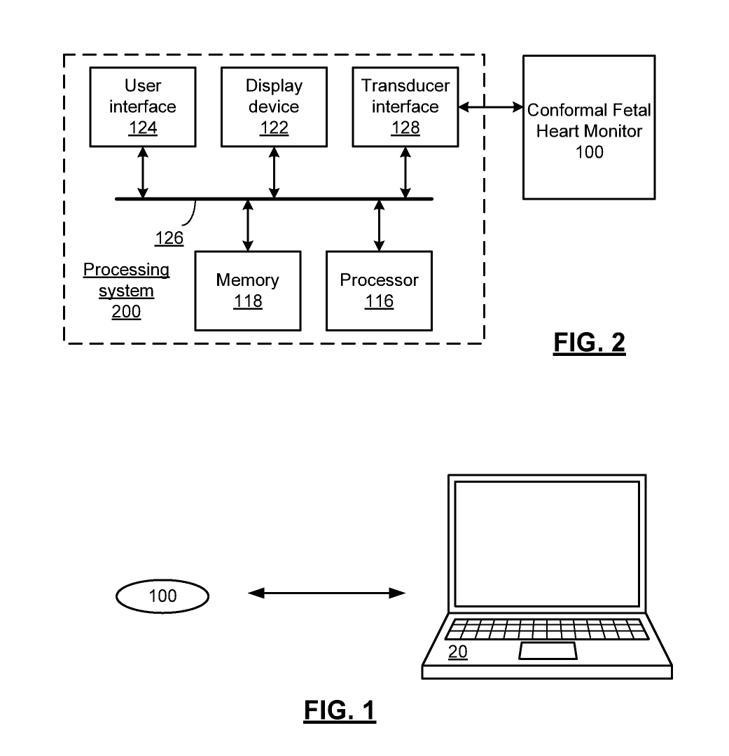

FIG. 1 is a block diagram representation of an embodiment of fetal heart monitoring system including conformal fetal heart monitor 100 and personal computer 20 in accordance with the present invention. The conformal fetal heart monitor 100 includes an ultrasonic transducer for interrogation of the fetal heart through the walls of the abdomen and the amniotic fluid of mother. A processing system, such as personal computer, embedded system computer, microcontroller, or existing monitoring equipment, 20 processes signals from the ultrasonic transducer and displays the data produced therefrom. In particular, the fetal heartbeat is monitored, based on the displayed heart beat signals in order to monitor the relative health of the fetus. While shown as being coupled to a personal computer 20, the conformal fetal heart monitor 100 can be mated to an existing monitoring workstation, or implemented in conjunction with a computing device with another portable display such as a tablet, phone, personal digital assistant, etc.

The conformal fetal heart monitor 100 is a vastly improved FHM with a soft and flexible housing that conforms to the abdomen of the patient. The conformal fetal heart monitor 100 improves patient care by improving patient comfort compared to rigid probes, and by better maintaining positioning on the abdomen even during movement of the patient, therefore allowing the patient improved freedom of movement and reducing the shifting of the transducer positioning and loss of the fetal heartbeat. This reduces the need for repositioning, and in turn reduces medical costs.

In an embodiment of the present invention, the conformal fetal heart monitor 100 incorporates wireless telemetry, including a lightweight battery-operated system, eliminating the need for cabling to the fetal monitor. The light weight and flexibility of the conformal FHM will also enable the use of adhesion methods that can eliminate the use of adjustable straps. Together, these innovations significantly improve patient comfort, acceptance, mobility, and satisfaction, and reduce alarm rates and labor costs associated with patient monitoring.

Further details regarding the operation of the fetal heart monitoring system will be described in greater detail in conjunction with FIGS. 2-8, which further describe several optional functions and features.

FIG. 2 is a block diagram representation of an embodiment of fetal heart monitoring system including conformal fetal heart monitor 100 and processing system 200 in accordance with the present invention. The processing system 200 includes a processor or microcontroller 116, memory 118, transducer interface 128 that includes low noise RF and baseband analog electronics, display device 122, and user interface 124 that are coupled via bus 126. While a particular bus architecture is shown that includes bus 126, other architectures with additional direct connectivity between modules or with additional buses, both wired and wireless, are likewise possible. As discussed in conjunction with FIG. 1, the processing system 200 can be implemented via a personal computer 20, an existing monitoring workstation, or implemented in conjunction with a computing device with another portable display such as a tablet, phone, personal digital assistant, etc.

Transducer interface 128 optionally includes one or more drivers for generating transducer signaling that causes the conformal fetal heart monitor 100 to produce an ultrasonic transmission signal. In an embodiment of the present invention, the conformal fetal heart monitor 100 includes a piezoelectric transducer array that produces ultrasonic waves, such as in the range of 100 kHz to 5 MHz. These ultrasonic waves travel through the wall of the abdomen and the amniotic fluid and are partially reflected by the tissue of the fetus to produce echo returns. The conformal fetal heart monitor 100 generates an ultrasonic receive signal in response to the ultrasonic echo return signal. The interrogation signal is reflected off the fetus' heart walls and the return signal is a Doppler-shifted copy of the original, where the frequency shift is proportional to the heart wall velocity. The ultrasonic receive signal is down converted to base bad, amplified, and then sampled to produce a digital data stream by the transducer interface 128 for processing by processor 116 in conjunction with corresponding beam angle data.

In particular, the phase shift between received signals reflecting from the heart of the fetus indicates the heart rate of the fetus. Additional measurements can be taken, for example, the time it takes for an echo return to be received from the time it was transmitted indicates the depth of the structure that produced the echo. While fetal heart monitoring systems typically range gate to isolate the fetus' heart, the processing system can interrogate the entire volume and then employ range gating if desired. In addition, the strength of the echo can indicate the density of the structure that produced the echo. The transducer interface can further include low noise RF and baseband analog electronics a for driving signals, and a universal serial bus (USB) interface, Firewire interface, or other serial or parallel interface for coupling to the conformal fetal heart monitor 100. While shown as a separate device, portions of the transducer interface 128 can be incorporated in the fetal heart monitor 100 itself.

Processor 116 can be implemented using a shared processing device, individual processing devices, or a plurality of processing devices that operate in conjunction with memory 118. Such a processing device may be a microprocessor, micro-controller, digital signal processor, microcomputer, central processing unit, field programmable gate array, programmable logic device, state machine, logic circuitry, digital circuitry, and/or any device that manipulates signals based on operational instructions. The memory 118 may be a single memory device or a plurality of memory devices. Such a memory device may be a read-only memory, random access memory, volatile memory, non-volatile memory, static memory, dynamic memory, flash memory, and/or any device that stores digital information. Note that when the processor 116 implements one or more of its functions via a state machine, digital circuitry, and/or logic circuitry, the memory storing the corresponding operational instructions is embedded with the circuitry comprising the state machine, digital circuitry, and/or logic circuitry.

In an embodiment of the present invention, the memory 118 stores an application such as a fetal heart monitoring application that is executed by the processor 116. User interface 222 includes user input devices such as a touch screen, one or more buttons, speakers and/or other user interface devices to be used, in conjunction with display device 122 to receive user input and to provide output to the user in order to control the operation of the fetal heart monitoring application. In operation, the fetal heart monitoring application analyzes the ultrasonic receive data to produce a readout with the heart rate trace and/or numerical value for the heart beat or a visual or audible representation of the heart beat for display on the display device 122, and further to detect and analyze the fetal heartbeat. The maternal heart beat can also be displayed on the display device 122. Display device 122 can include a printer, liquid crystal display, a light emitting diode display, a plasma display, a projection display, an audio speaker, or other display device. An audio or visual alarm can be incorporated, and is used in the event that the fetal heart beat significantly changes or is lost.

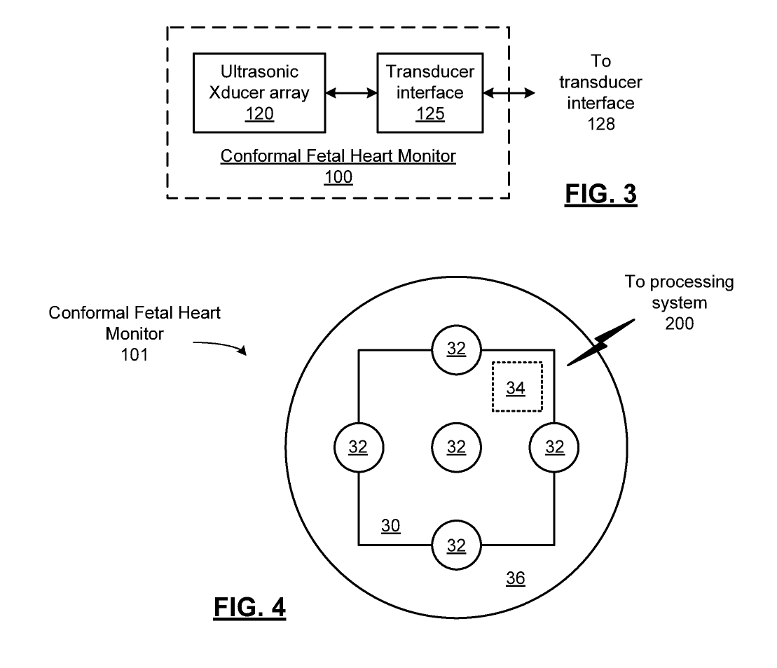

FIG. 3 is a block diagram representation of a conformal fetal heart monitor 100 in accordance with the present invention. In particular, the conformal fetal heart monitor 100 includes an ultrasonic transducer array that generates multiple ultrasonic transmission signals, that receives an ultrasonic return signal (echo), and that generates an ultrasonic receive signal in response to the ultrasonic return signal. A transducer interface 125 operates in conjunction with transducer interface 128 to couple the ultrasonic receive signal to a remote processing system, as either a digital signal or an analog signal.

Each fetal transducer can be equipped with and utilizes appropriate control circuitry such that each unit is part of a mesh network, providing communication either via wire, fiber optic, radio signal, or light signal between units. Such mesh network enables all of the units (an array) either within a physical plant (local area network) or outside of a physical plant (wide area network) to communicate with each other. Software revisions can be sent via wire, light, or radio to a single unit, and this unit can pass the software revision to each successive unit within the array, be it in a local area network within a structure or outside of the structure in a wide area network. Battery status, working status, temperature, time of operation, out of range alarm, cycle counts, physiological values, etc., may be sent along the mesh network to a plurality of central monitoring stations within a local area network or wide area network, such that all of these parameters can be monitored even though the unit of interest within the array of units is outside of the radio range of the monitoring station.

The mesh network can be built upon an array connected via wire, radio signal, light signal, fiber optic signal, etc. Each unit within the array has a unique electronic address. There may or may not be a master unit, and each unit can be identical. Hence, each unit in the array may assume a control function if deemed necessary by the programmer, although a master unit is not necessary for the mesh network to function properly. As discussed in conjunction with FIG. 2, the transducer interface 125 optionally includes one or more drivers for generating transducer signaling that causes the ultrasonic transducer array 120 to produce ultrasonic transmission signals. In an embodiment of the present invention, the ultrasonic transducer array 120 produces ultrasonic waves, such as in the range of 100 kHz to 5 MHz. These ultrasonic waves travel through the wall of the abdomen and the amniotic fluid and are partially reflected by the tissue of the fetus to produce echo returns. The ultrasonic transducer array 120 receives a reflected ultrasonic signal in response to the ultrasonic echo return signal. The ultrasonic receive signal is amplified sampled and converted to digital data by the transducer interface 125 or 128 for processing by processor 116.

The fetal heart monitor may have any number of transducer elements, arranged in any of a number of patterns. In one embodiment, the transducer has seven elements, with one central transmitting/receiving element and six transmitting/receiving elements arranged radially around the central element. In another embodiment, the transducer has nine elements, with one central transmitting/receiving element and eight transmitting/receiving elements arranged radially around the central element. Several additional embodiments are possible.

FIG. 4 is a top view of an embodiment of conformal fetal heart monitor 101 in accordance with the present invention. A conformal fetal heart monitor 101 is shown that is an embodiment of conformal fetal heart monitor 100. The conformal fetal heart monitor 101 includes one or more circuit boards 30 that supports an electrical matching network, wireless transducer interface, processing circuitry, power, and/or other circuit components. The circuit board or circuit boards may have varying dimensions, shapes, or thicknesses, depending on the specific layout of the fetal heart monitor and transducer array elements.

The electrical matching network is required to match the source impedance of the RF oscillator to the load impedance of the piezoelectric transducer for maximum energy transfer efficiency. The processing electronics may be used to beam form, modulate or turn on/off individual or groups of elements, convert the RF signal to baseband, amplify the detected signal, and digitize the processed data. The wireless transducer interface 34 includes a driver for driving the ultrasonic transducer array, amplifiers for amplifying generating an analog ultrasonic receive signal and an analog to digital converter for converting the ultrasonic receive signal to ultrasonic receive data. The wireless transducer interface 34 further includes a lightweight battery operated wireless transceiver that transmits the ultrasonic receive data to the remote processing system 200. In this embodiment, wireless transceiver of wireless transducer interface 34 and the transducer interface 128 of processing system 200 each operate in conjunction with a wireless communication protocol such as a Bluetooth communication protocol, a Zigbee communication protocol, an 802.11 communication protocol or other short range wireless communication protocol, either standard or proprietary. The ultrasound transducer array elements and/or electrical circuit components can be electrically connected to circuit boards using any of several methods, including soldering, silver conductive ink, conductive epoxy, wire bonding, cabling, or other means.

The conformal fetal heart monitor 101 further includes a flexible housing 36 that holds or connects with the circuit board or circuit boards 30, the ultrasonic transducer array 120 and the transducer interface 30. This housing may also serve as a substrate for the ultrasound transducer array elements. The flexible housing can be constructed of a silicone rubber, urethane, or other flexible or elastic materials that, when deployed, conforms to the surface of the abdomen of a patient. The flexible housing material may have varying degrees of stiffness to enhance flexibility, or varying degrees of tackiness to enhance adhesion to the patient's body. The flexible housing may be pre-strained so as to have a resting curvature that best conforms to the body. A rigid housing may be connected to the flexible housing and/or house the circuit board or boards and circuit board components. In one embodiment, the rigid housing is made from plastic. As discussed in conjunction with the conformal fetal heart monitor 100 of FIG. 1, the conformal fetal heart monitor 101 improves patient care by improving patient comfort compared to rigid probes, and by better maintaining positioning on the abdomen even during movement of the patient, therefore allowing the patient improved freedom of movement and reducing the shifting of the transducer positioning and loss of the fetal heartbeat. This reduces the need for repositioning, and in turn reduces errors and medical costs.

In practice, fetal heart monitors are typically used in conjunction with a tocodynamometer, also referred to as a tocotransducer or toco. The toco is a separate device that is strapped to the patient and incorporates a pressure sensor to measure uterine contractions. The proposed conformal fetal heart monitor device may also feature an integrated toco, or a plurality of integrated tocos. This embodiment can enable simultaneous measurement of the fetal heart beat, maternal heartbeat, and uterine contractions in a single device. In this embodiment, pressure sensors or other uterine contraction measurement devices are incorporated adjacent to the ultrasound transducer array, within the ultrasound transducer array, or in the transducer housing.

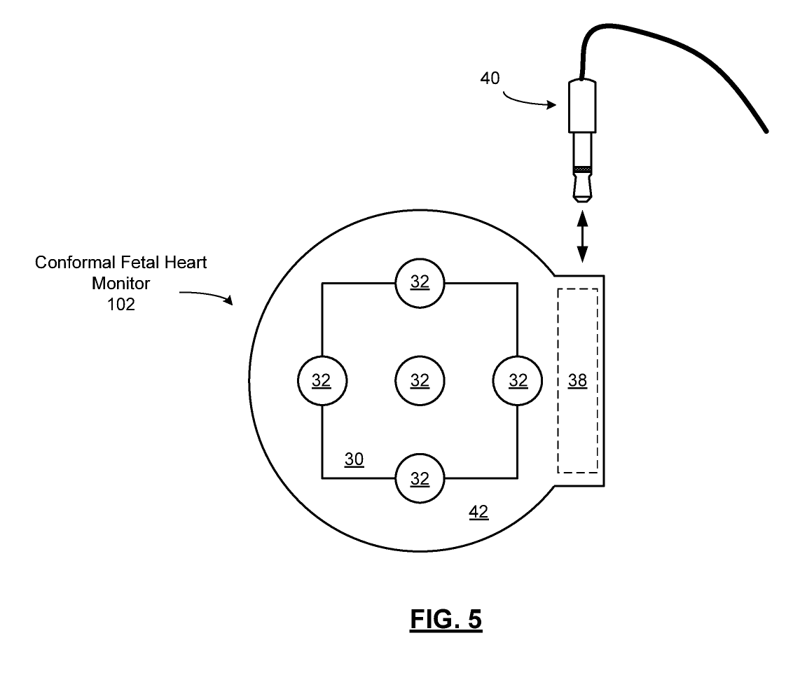

FIG. 5 is a top view of an embodiment of conformal fetal heart monitor 102 in accordance with the present invention. A conformal fetal heart monitor 102 is shown that is another embodiment of conformal fetal heart monitor 100. In particular, a conformal fetal heart monitor 102 is shown that includes similar elements to conformal fetal heart monitor 101 that are referred to by common reference numerals. In this embodiment however, a wired transducer interface 38 is included having a driver for driving the ultrasonic transducer array and for converting the ultrasonic receive signal to ultrasonic data in addition to a wired interface that couples the ultrasonic receive data to the processing system 200.

As shown, the wired transducer interface 38 includes a jack and the transducer interface 128 of processing system 200 operated in a conjunction with a plug, such as miniature or subminiature phone plug 40. Other plug and jack configurations are likewise possible including configuration where the wired transducer interface 38 includes a plug for coupling to a jack of transducer interface 128 or processing system 200 and further including other types of plugs and/or jacks. In another embodiment of the present invention, the wired transducer interface 38 and transducer interface 138 each include a universal serial bus (USB) interface for coupling the conformal fetal heart monitor 102 to a personal computer, tablet PC, or other processing device 20.

In addition, the plug or jack can be located on the top of the housing rather than the side as shown, the plug or jack coupling can be coaxial to the centroid of the housing 42 or aligned in other orientations. Further, a terminal and clip arrangement or other connector configurations can be used in place of a plug and jack.

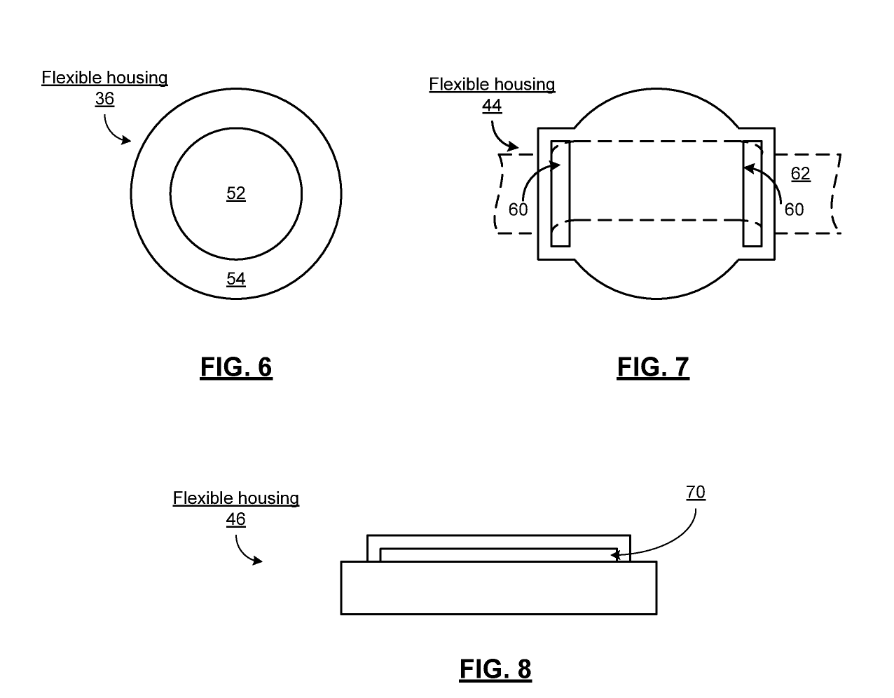

FIG. 6 is a back view of an embodiment of a flexible housing 36 in accordance with the present invention. In this embodiment, the flexible housing 36 includes an adhesive front that is exposed upon peeling off a non-stick protective sheet. The adhesive front includes an adhesive outer region 54 for adhering to the abdomen of the patient and further a non-adhesive inner region 52. The non-adhesive inner region 52 is configured to correspond to the positioning of piezoelectric transducers 32 of ultrasonic transducer array 120.

When deployed, a liquid or gel coupling can be applied to the non-adhesive inner region 52. The non-adhesive inner region 52 can be optionally recessed from the adhesive outer region 54 for this purpose. The liquid or gel coupling provides an ultrasonic coupling of ultrasonic waves, to and from the ultrasonic transducer array 120. The adhesive outer region 54 secures the flexible housing 36 of the conformal fetal heart monitor 100, 101 or 102 to the skin of the abdomen. While flexible housing 36 is shown, the shape of the flexible housing in this embodiment can likewise conform with the shape of flexible housing 42 or other flexible housing shapes.

FIG. 7 is a top view of another embodiment of a flexible housing 44 in accordance with the present invention. In this embodiment, the flexible housing 44 operates in a similar fashion as flexible housing 36, but includes channels 60 for receiving a strap 62. The strap 62 is threaded through the channels 60 to encircle the abdomen of the patient and secure the conformal fetal heart monitor 100 to 100, 101 or 102 to abdomen of the patient. Alternate methods include a strap attached to a button at the top of the fetal heart monitor, an elastic band around the waist of the patient, Velcro straps, light vacuum pressure between the skin and fetal heart monitor, external pneumatic pressure, or other techniques.

When deployed, a liquid or gel coupling can be applied to the back of flexible housing 44. The gel coupling provides an ultrasonic coupling of ultrasonic waves, to and from the ultrasonic transducer array 120 and to and from the mother's abdomen. The strap secures the flexible housing 44 of the conformal fetal heart monitor 100 to the skin of the abdomen. While flexible housing 44 is shown, the shape of the flexible housing in this embodiment can likewise conform with the shape of flexible housing 42 to further include a plug or jack connection and/or to conform with other flexible housing shapes.

FIG. 8 is a side view of another embodiment of a flexible housing 36 or 42 in accordance with the present invention. In this embodiment, the flexible housing 46 operates in a similar fashion as flexible housing 36, but includes a channel 70 along the top of the housing for receiving a strap (not specifically shown). The strap is threaded through the channel 70 to encircle the abdomen of the patient and secure the conformal fetal heart monitor 100 to 100, 101 or 102 to abdomen of the patient. Alternatively, a rigid housing may be used to receive a strap, such as one made of plastic. This rigid housing would be connected to a lower flexible housing.

When deployed, a liquid or gel coupling can be applied to the front of flexible housing 46. The gel coupling provides an ultrasonic coupling of ultrasonic waves, to and from the ultrasonic transducer array 120. The adhesive or strap secures the flexible housing 46 of the conformal fetal heart monitor 100 to the skin of the abdomen. While flexible housing 46 is shown, the shape of the flexible housing in this embodiment can likewise conform with the shape of flexible housing 42 to further include a plug or jack connection and/or to conform with other flexible housing shapes.

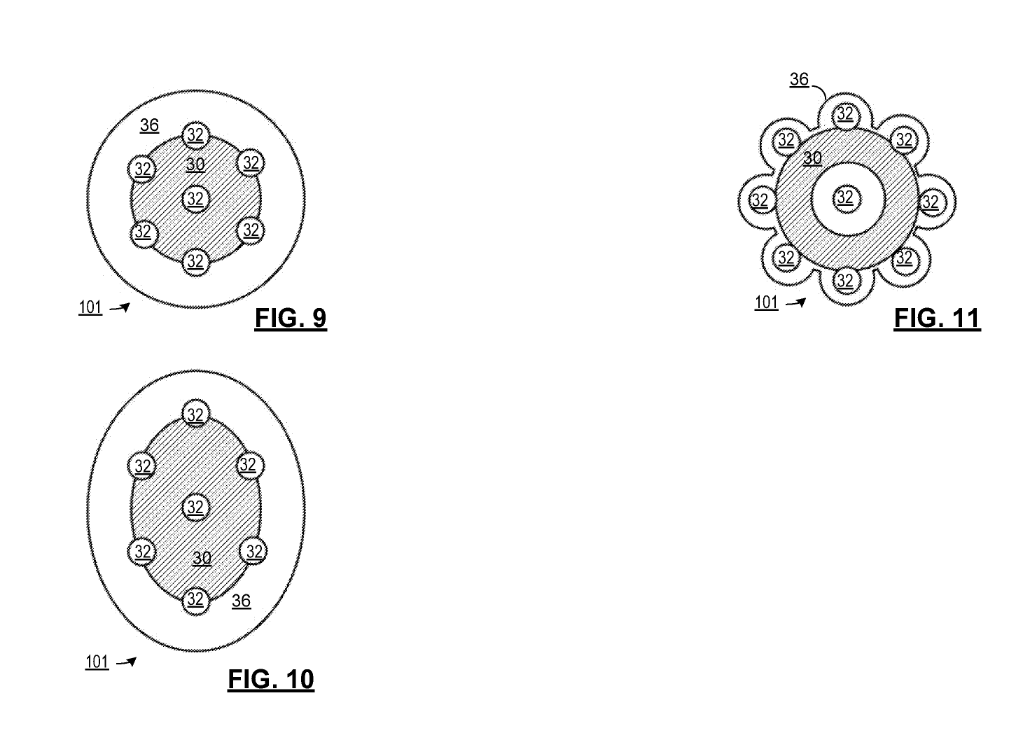

FIGS. 9-11 are top views of other embodiments of conformal fetal heart monitor 101 in accordance with the present invention. As discussed in conjunction with FIG. 3, the fetal heart monitor may have any number of transducer elements 32, arranged in any of a number of patterns. In particular, several configurations are shown that are similar to the embodiment of FIG. 4 and that use common reference numerals to refer to similar elements. In the embodiment of FIG. 9, the transducer has seven elements 32, with one central transmitting/receiving element and six transmitting/receiving elements arranged radially around the central element. In other embodiments shown in conjunction with FIGS. 10 and 11, the transducer has seven or nine elements 32, with one central transmitting/receiving element 32 and six or eight transmitting/receiving elements 32 arranged radially around the central element 32.

As may also be used herein, the term(s) “coupled to” includes direct connection between items and/or indirect connection between items via an intervening item (e.g., an item includes, but is not limited to, a component, an element, a circuit, and/or a module).

The present invention has also been described above with the aid of method steps illustrating the performance of specified functions and relationships thereof. The boundaries and sequence of these functional building blocks and method steps have been arbitrarily defined herein for convenience of description. Alternate boundaries and sequences can be defined so long as the specified functions and relationships are appropriately performed. Any such alternate boundaries or sequences are thus within the scope and spirit of the claimed invention.

The present invention has been described above with the aid of functional building blocks illustrating the performance of certain significant functions. The boundaries of these functional building blocks have been arbitrarily defined for convenience of description. Alternate boundaries could be defined as long as the certain significant functions are appropriately performed. Similarly, flow diagram blocks may also have been arbitrarily defined herein to illustrate certain significant functionality. To the extent used, the flow diagram block boundaries and sequence could have been defined otherwise and still perform the certain significant functionality. Such alternate definitions of both functional building blocks and flow diagram blocks and sequences are thus within the scope and spirit of the claimed invention. One of average skill in the art will also recognize that the functional building blocks, and other illustrative blocks, modules and components herein, can be implemented as illustrated or by discrete components, application specific integrated circuits, processors executing appropriate software and the like or any combination thereof.

The present invention has been described in conjunction with various illustrative embodiments that include many optional functions and features. It will be apparent to those skilled in the art that the disclosed invention may be modified in numerous ways, the functions and features of these embodiments can be combined in other embodiments not expressly shown, and may assume many embodiments other than the preferred forms specifically set out and described above. Accordingly, it is intended by the appended claims to cover all modifications of the invention which fall within the true spirit and scope of the invention.

What is claimed is:

1. A conformal fetal heart monitor comprising:
a flexible substrate;
a ultrasonic transducer array, coupled to the flexible substrate, that generates a ultrasonic transmission signal, that receives an ultrasonic return signal, and that generates an ultrasonic receive signal in response to the ultrasonic return signal;
a transducer interface, coupled to the ultrasonic transducer array, wherein the transducer interface includes:
a driver for driving the ultrasonic transducer array and that converts the ultrasonic receive signal to ultrasonic receive data; and
a wireless transceiver that wirelessly transmits the ultrasonic receive data to the remote processing system; and
a flexible housing that houses the flexible substrate, the ultrasonic transducer array and the transducer interface, wherein the flexible housing flexibly, when deployed, conforms to the surface of an abdomen of a patient and.

2. The conformal fetal heart monitor of claim 1 wherein the flexible housing includes at least one of: a silicone rubber element and a polyurethane element.

3. The conformal fetal heart monitor of claim 1 wherein the flexible housing includes an adhesive front for adhering to the abdomen of the patient and wherein the adhesive front includes a non-adhesive inner region for application of a gel coupling.

4. A conformal fetal heart monitor comprising:
a flexible substrate;
an ultrasonic transducer array, coupled to the flexible substrate, that generates a ultrasonic transmission signal, that receives an ultrasonic return signal, and that generates an ultrasonic receive signal in response to the ultrasonic return signal;
a transducer interface, coupled to the ultrasonic transducer array, wherein the transducer interface includes:
a driver for driving the ultrasonic transducer array and that converts the ultrasonic receive signal to ultrasonic data; and
a wired interface that couples the ultrasonic receive data to the remote processing system; and
a flexible housing that houses the flexible substrate, the ultrasonic transducer array and the transducer interface, wherein the flexible housing flexibly, when deployed, conforms to the surface of an abdomen of a patient and wherein the flexible housing includes an adhesive front for adhering to the abdomen of the patient.

5. The conformal fetal heart monitor of claim 4 wherein the wired interface includes at least one of: a plug and a jack.

6. The conformal fetal heart monitor of claim 4 wherein the wired interface includes a universal serial bus interface.

7. The conformal fetal heart monitor of claim 4 wherein the flexible housing includes at least one of: a silicone rubber element and a polyurethane element.

8. The conformal fetal heart monitor of claim 4 wherein the adhesive front includes a non-adhesive inner region for application of a gel coupling.

9. A conformal fetal heart monitor comprising:
a flexible substrate;
a ultrasonic transducer array, coupled to the flexible substrate, that generates a ultrasonic transmission signal, that receives an ultrasonic return signal, and that generates an ultrasonic receive signal in response to the ultrasonic return signal;
a transducer interface, coupled to the ultrasonic transducer array, that couples the ultrasonic receive signal to a remote processing system; and
a flexible housing that houses the flexible substrate, the ultrasonic transducer array and the transducer interface, wherein the flexible housing flexibly, when deployed, conforms to the surface of an abdomen of a patient and wherein the flexible housing includes an adhesive front for adhering to the abdomen of the patient.

10. The conformal fetal heart monitor of claim 9 wherein the transducer interface includes:
a driver for driving the ultrasonic transducer array and that converts the ultrasonic receive signal to ultrasonic receive data; and
a wireless transceiver that couples the ultrasonic receive signal to the remote processing system by wireless transmitting the ultrasonic receive data to the remote processing system.

11. The conformal fetal heart monitor of claim 9 wherein the transducer interface includes:
a driver for driving the ultrasonic transducer array and that converts the ultrasonic receive signal to ultrasonic data; and
a wired interface that couples the ultrasonic receive data to the remote processing system.

12. The conformal fetal heart monitor of claim 11 wherein the wired interface includes at least one of: a plug and a jack.

13. The conformal fetal heart monitor of claim 11 wherein the wired interface includes a universal serial bus interface.

14. The conformal fetal heart monitor of claim 9 wherein the flexible housing includes at least one of: a silicone rubber element and a polyurethane element.

15. The conformal fetal heart monitor of claim 9 wherein the adhesive front includes a non-adhesive inner region for application of a gel coupling.
